(19) United States
(12) Patent Application Publication
(10) Pub. No.: US 2023/0204544 A1
(43) Pub. Date: Jun. 29, 2023

(54) CALIBRATION METHODS FOR MASS SPECTROMETRY MEASUREMENTS

(71) Applicant: Mayo Foundation for Medical Education and Research, Rochester, MN (US)

(72) Inventors: Stefan K.G. Grebe, Rochester, MN (US); Jennifer V. Kemp, Rochester, MN (US); Anthony D. Maus, Rochester, MN (US); Steven Lewis Ramsay, Victoria (AU)

(21) Appl. No.: 17/926,807

(22) PCT Filed: May 21, 2021
(86) PCT No.: PCT/US2021/033569

§ 371 Date: Nov. 21, 2022

Related U.S. Application Data
(60) Provisional application No. 63/028,332, filed on May 21, 2020.

(51) Int. Cl. G01N 30/72; G01N 27/623; G01N 33/58

(52) U.S. Cl. CPC G01N 30/7233; G01N 27/623; G01N 33/58; G01N 2030/027

(57) ABSTRACT

Provided herein are single-substance multi-point calibration techniques for quantitative mass spectrometry using the theoretical relative abundance of isotopes in a calibrator. Also provided herein are methods of determining the concentration of an analyte using a calibrator for the analyte.

### CROSS-REFERENCE TO RELATED APPLICATIONS

This application claims the benefit of U.S. Provisional Application No. 63/028,322, filed May 21, 2020, the contents of which are hereby incorporated by reference.

### TECHNICAL FIELD

This disclosure is related to internal calibration techniques for quantitative mass spectrometry. Such techniques include single-substance multi-point calibration.

### BACKGROUND

Liquid chromatography (LC)-mass spectrometry (MS) has emerged as a powerful analysis tool in the clinical laboratory. Quantitative results are most commonly produced by adding an isotopically labeled internal standard (IS) to each sample for correction of various measurement process mishaps, ionization irregularities, and instrument performance changes during a measurement run, and then applying an external calibration curve to the IS corrected result. However, this typically requires daily creation, and measurement of, an accurate and consistent (usually multipoint) concentration calibration curve over the life cycle of a clinical test. This can be a laborious, time consuming, and costly process. It can also be challenging to maintain over extended time periods. New calibration methods that simplify the calibration process are desirable.

### SUMMARY

Provided herein are methods for determining the concentration of an analyte in a sample comprising the analyte and one or more calibrators for each analyte, wherein the concentration of at least one of the one or more calibrators for each analyte is known, the method comprising: detecting the analyte and at least one of the one or more the calibrators for the analyte in the sample using a mass spectrometry (MS) technique (e.g., a MS, MS/MS, or MSⁿ technique); and determining the concentration of the analyte in the sample based on two or more measured peaks in the isotopic distribution of at least one of the one or more calibrators for the analyte.

In some embodiments, the calibrator for the analyte is isotopically labeled. In some embodiments, the calibrator for the analyte is not isotopically labeled.

In some embodiments, determining the concentration of the analyte comprises generating a calibration curve based on: a) the known concentration of the calibrator for the analyte; b) the theoretical relative abundance of the isotopes that correspond to the two or more measured peaks from the isotopic distribution of the calibrator for the analyte; and c) the signal intensity of the two or more measured peaks in the isotopic distribution of the calibrator for the analyte, wherein the known concentration of the calibrator for the analyte and the theoretical relative abundance of the isotopes that correspond to the two or more measured peaks in the isotopic distribution of the calibrator for the analyte are multiplied together to generate a calculated concentration for each of the isotopes that correspond to the two or more measured peaks from the isotopic distribution of the calibrator for the analyte.

In some embodiments, determining the concentration of the analyte comprises generating a calibration curve based on: a) the known concentration of the calibrator for the analyte; b) the theoretical relative abundance (or the experimentally derived relative abundance, or both the theoretical relative abundance and the experimentally derived relative abundance) of the isotopes that correspond to the two or more measured peaks from the isotopic distribution of the calibrator for the analyte; and c) the signal intensity of the two or more measured peaks in the isotopic distribution of the calibrator for the analyte, wherein the known concentration of the calibrator for the analyte and the theoretical relative abundance (or the experimentally derived relative abundance, or both the theoretical relative abundance and the experimentally derived relative abundance) of the isotopes that correspond to the two or more measured peaks in the isotopic distribution of the calibrator for the analyte are multiplied together to generate a calculated concentration for each of the isotopes that correspond to the two or more measured peaks from the isotopic distribution of the calibrator for the analyte. For example the calibration curve can be based on: a) the known concentration of the calibrator for the analyte; b) the experimentally derived relative abundance, i.e., the empirically derived relative abundance, of the isotopes that correspond to the two or more measured peaks from the isotopic distribution of the calibrator for the analyte; and c) the signal intensity of the two or more measured peaks in the isotopic distribution of the calibrator for the analyte.

In some embodiments, determining the concentration of the analyte comprises generating a calibration curve based on: a) the known concentration of the calibrator for the analyte; b) the theoretical relative abundance (or the experimentally derived relative abundance, or both the theoretical relative abundance and the theoretical relative abundance) of the isotopes that correspond to the two or more measured peaks from the isotopic distribution of the calibrator for the analyte; and c) the signal intensity of the two or more measured peaks in the isotopic distribution of the calibrator for the analyte, wherein the known concentration of the calibrator for the analyte and the theoretical relative abundance (or the experimentally derived relative abundance, or both the theoretical relative abundance and the theoretical relative abundance) of the isotopes that correspond to the two or more measured peaks in the isotopic distribution of the calibrator for the analyte are multiplied together to generate a calculated concentration for each of the isotopes that correspond to the two or more measured peaks from the isotopic distribution of the calibrator for the analyte.

In some embodiments, the calibration curve from the calibrator for the analyte is applied to at least one measured peak in the isotopic distribution of the analyte by performing a linear regression analysis. In some embodiments, the linear regression analysis comprises solving the formula:

$$\frac{( Int - b ) m}{Theorectical Rel . Abun . of Isotope}$$

wherein Int is the signal intensity from a measured peak in the isotopic distribution of the analyte;
m is the slope of the calibration curve from the calibrator for the analyte; and
b is the y-intercept of the calibration curve from the calibrator for the analyte, for at least one measured peak in the isotopic distribution of the analyte to yield an isotope concentration in the analyte.

In some embodiments, the linear regression analysis comprises solving the formula:

$$\frac{( Int - b ) m}{Exp . Rel . Abun . of Isotope}$$

wherein Int is the signal intensity from a measured peak in the isotopic distribution of the analyte;
m is the slope of the calibration curve from the calibrator for the analyte; and
b is the y-intercept of the calibration curve from the calibrator for the analyte, for at least one measured peak in the isotopic distribution of the analyte to yield an isotope concentration in the analyte.

In some embodiments, the calibration curve from the calibrator for the analyte is applied to at least one measured peak in the isotopic distribution of the analyte using a polynominal curve fit. For example, a polynomial curve fit can be used when the instrument response is not linear over the entire measurement range.

In some embodiments, the calibration curve from the calibrator for the analyte is applied to two or more measured peaks in the isotopic distribution of the analyte to yield two or more isotope concentrations for the analyte. In some embodiments, the calibration curve from the calibrator for the analyte is applied to three or more measured peaks in the isotopic distribution of the analyte to yield two or more isotope concentrations for the analyte. In some embodiments, the calibration curve from the calibrator for the analyte is applied to five or more measured peaks in the isotopic distribution of the analyte to yield two or more isotope concentrations for the analyte. In some embodiments, the calibration curve from the calibrator for the analyte is applied to ten or more measured peaks in the isotopic distribution of the analyte to yield two or more isotope concentrations for the analyte.

In some embodiments, each isotope concentration for the analyte is averaged to yield the concentration of the analyte.

In some embodiments, the analyte is a polypeptide, a protein, a peptide, a small molecule, a polysaccharide, a lipid, a glycan, a dendrimer, or a nucleic acid. In some embodiments, the analyte is a steroid. In some embodiments, the analyte is a drug. In some embodiments, the calibrator for the analyte is a polypeptide, a protein, a peptide, a small molecule, a polysaccharide, a lipid, a glycan, a dendrimer, or a nucleic acid. In some embodiments, the calibrator for the analyte is a steroid. In some embodiments, the calibrator for the analyte is a drug.

In some embodiments, the calibrator for the analyte is a variant of the analyte.

In some embodiments, the calibrator for the analyte has a different mass to charge ratio than the analyte. In some embodiments, the calibrator for the analyte has similar physical and/or chemical properties as the analyte. In some embodiments, the calibrator for the analyte has similar ionization properties as the analyte.

In some embodiments, the calibrator for the analyte is a polypeptide, and the analyte is a polypeptide. In some embodiments, the calibrator for the analyte is a single amino acid variant of the analyte.

In some embodiments, the concentration of two or more analytes in the sample is determined. In some embodiments, the concentration of five or more analytes in the sample is determined.

In some embodiments, wherein the concentration of two or more analytes in the sample is determined, the calibrator for the analyte for two or more of the analytes is the same. In some embodiments, wherein the concentration of five or more analytes in the sample is determined, the calibrator for the analyte for two or more of the analytes is the same.

In some embodiments, the MS technique comprises the use of liquid chromatography (LC) or gas chromatography (GC). In some embodiments, the MS technique comprises an LC-MS technique, a GC-MS technique, or a MALDI-MS technique. In some embodiments, the MS technique comprises mass-spectrometry (e.g., single-stage mass spectrometry), tandem mass spectrometry (MS/MS), and sequential mass spectrometry (MSⁿ). In some embodiments, the mass spectrometry technique comprises the use of a single quadrupole mass spectrometer (Q), a triple quadrupole (QQQ) mass spectrometer, a linear ion trap mass spectrometer, a Q-linear ion trap mass spectrometer, a time of flight (TOF) mass spectrometer, a quadrupole-time of flight (Q-TOF) mass spectrometer, or a trapping mass spectrometer (Orbitrap or FTICR). In some embodiments, the MS technique comprises the use of high-resolution accurate mass-mass spectrometry (HRAM-MS).

In some embodiments, the MS technique is performed on lower resolution instruments for low molecular weight compounds.

In some embodiments, the sample is de-proteinized before detecting the analyte and the calibrator for the analyte in the sample using a mass spectrometry (MS) technique. In some embodiments, the sample is de-proteinized before the calibrator for the analyte is added to the sample. In some embodiments, the sample is de-proteinized after the calibrator for the analyte is added to the sample. In some embodiments, the de-proteinization comprises precipitation of one or more polypeptides in the sample. In some embodiments, the de-proteinization comprises exposing the sample to acidified ethanol.

In some embodiments, the sample is a biological sample. In some embodiments, the biological sample is a blood, urine, lachrymal, plasma, serum, or saliva sample. In some embodiments, the sample is from a mammal. In some embodiments, the mammal is a human.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 depicts the general process for single-substance multi-point calibration.

FIG. 2 is a graph depicting an example calibration curve.

FIG. 3 is a graph depicting a comparison between the experimental and the theoretical isotopic distribution for ¹⁵N-labeled IGF-1.

FIG. 4 is a graph depicting a comparison between the IGF-1A70T variant and wild-type IGF-1.

FIG. 5 is a table containing results of the single-substance multi-point calibration data compared to the reference test results.

FIG. 6A is a graph depicting the Weighted Deming regression of patient results using single-substance multi-point calibration as compared to a reference method.

FIG. 6B is a graph depicting the Deming regression of patient results using single-substance multi-point calibration as compared to a reference method.

FIG. 6C is a graph depicting the Passing-Bablok regression of patient results using single-substance multi-point calibration as compared to a reference method.

FIG. 6D is a graph depicting the linear regression analysis of patient results using single-substance multi-point calibration as compared to a reference method.

FIG. 6E is a graph depicting the weighted linear regression of patient results using single-substance multi-point calibration as compared to a reference method.

FIG. 7 is a table of fitting parameters and statistics for determining the goodness of fit when using multiple linear regression analyses.

FIGS. 8A and 8B are graphs depicting the application of single-substance multi-point calibration linear regression analysis correction to experimentally-derived spectra. FIG. 8A shows an overlay of isotopic distribution of the medium quality control level (n=20). FIG. 8B shows an overlay of isotopic distribution of medium quality control after linear regression correction (n=20).

FIG. 9 depicts the theoretical positive ion mode mass spectra for two low molecular weight isotopically labeled internal standards (IS): cortisol labeled with four deuterium atoms (left), and cortisone labeled with seven deuterium atoms (right).

FIG. 10 shows a comparison of cortisol measurement results for various samples across the analytical range (0-20 μg/dL) with a seven-point calibration curve (blank plus six calibrators). There is excellent agreement for the sample results between the two calibration approaches.

FIG. 11 are plots showing the process for determining relative abundance by averaging replicate measurements (n=100).

FIG. 12 is a plot showing the comparison of patient results obtained by traditional external calibration (x-axis) with corresponding ¹⁵N-calibrator calibration (y-axis).

FIG. 13 depicts the overlaid (positive ion mode) experimental and theoretical positive ion mode mass spectra for deuterium labeled cortisol (left) and cortisone (right) calibrators, which show concordance.

FIGS. 14A-14C are plots showing comparisons of patient results obtained for simultaneous cortisol and cortisone quantification in urine samples by traditional external calibration (x-axes) with corresponding calibrator calibration results (y-axes). FIG. 14A and FIG. 10 show cortisol quantification, and FIGS. 14B and 14C show cortisone quantification.

FIG. 15 is a table showing the imprecision of replicate measurements of IGF-1 using two different molecules for single-substance multi-point calibration.

FIG. 16 is a graph depicting the linear regression analysis of patient results using the A70T variant as a single-substance multi-point calibration as compared to a reference method.

FIG. 17 is a table showing the imprecision of replicate measurements of cortisol and cortisone using two different molecules for single-substance multi-point calibration.

FIG. 18 is a plot showing the process for establishing the concentration relationship between the calibrator and an external calibration curve. First, the calibrator linear regression correction is applied to the reference calibrators to yield a concentration relative to the calibrator. This is replicated 3 times and the average relative concentration is plotted versus the established reference calibrator concentrations. The resulting linear regression equation is then applied to each control and patient sample to yield a final result. Once this linear regression equation has been generated, there is no need for external calibration curves.

### DETAILED DESCRIPTION

Materials and methods for single-substance multi-point calibration are provided. In some embodiments, provided herein are methods for determining the concentration of an analyte in a sample that comprises the analyte and a calibrator for the analyte. Such methods can include detecting the analyte and the calibrator for the analyte in the sample using a mass spectrometry (MS) technique and determining the concentration of the analyte based on two or more measured peaks in the isotopic distribution of the calibrator for the analyte in the sample. The concentration (e.g., in the sample) of the calibrator for the analyte is known.

In some embodiments, the concentrations of two or more analytes in the sample are determined. For example, the concentrations of three, four, five, six, seven, eight, nine, ten, eleven, twelve, thirteen, fourteen, fifteen, twenty or more analytes are determined.

In some embodiments, wherein the concentrations of two or more analytes (e.g., a first analyte, a second analyte, a third analyte, etc.) in the sample are determined, a different calibrator for the analyte can be used for each of the two or more analytes. In some embodiments, wherein the concentrations of two or more analytes (e.g., a first analyte, a second analyte, a third analyte, etc.) in the sample are determined, the same calibrator for the analyte can be used for two or more analytes. For example, the calibrator for the analyte for the first analyte can also be used as the calibrator for the analyte for the second analyte. In some embodiments, wherein the same calibrator for the analyte is used for two or more analytes, the analytes are chemically similar.

In some embodiments, determining the concentration of the analyte can include generating a calibration curve. Such calibration curves can be based on: the known concentration of the calibrator for the analyte; the theoretical relative abundance of the isotopes that correspond to the two or more measured peaks from the isotopic distribution of the calibrator for the analyte; and the signal intensity of the two or more measured peaks in the isotopic distribution of the calibrator for the analyte. As used herein the “theoretical relative abundance of an isotope” or “theoretical rel. abun. of isotope” refers to the average amount of the isotope that occurs naturally. The theoretical relative abundance of an isotope can be obtained through many databases and calculators including, but not limited to, Pacific Northwest National Laboratory Molecular Weight Calculator (see, omics.pnl.gov/software/molecular-weight-calculator on the World Wide Web); the database of Atomic Weights and Isotopic Compositions with Relative Atomic Masses by the National Institute of Standards and Technology (nist.gov/pml/atomic-weights-and-isotopic-compositions-relative-atomic-masses on the World Wide Web); and IsoBank (isobank.tacc.utexas.edu/en/ on the World Wide Web). In some embodiments, the calibration curve can be based on: the known concentration of the calibrator for the analyte; the empirically derived abundance of the isotopes that correspond to two or more peaks from the isotopic distribution of the calibrator for the analyte; and the signal intensity of the two or more measured peaks in the isotopic distribution of the calibrator for the analyte. The “empirically derived abundance of an isotope” or “experimentally derived abundance of an isotope” or “exp. rel. abun. of isotope” can be obtained through performing replicate measurements.

The known concentration of the calibrator for the analyte and the theoretical relative abundance of an isotope that corresponds to a measured peak in the isotopic distribution of the calibrator for the analyte can be multiplied together to generate a calculated concentration for the isotope, e.g., a concentration of the isotope in the calibrator for the analyte based on the concentration of the calibrator and either the average amount of the isotope that occurs naturally or the empirically derived abundance of the isotope. In some embodiments, the known concentration of the calibrator for the analyte and either the theoretical relative abundance of the isotopes or the empirically derived abundance of the isotopes that correspond to the two or more measured peaks in the isotopic distribution of the calibrator for the analyte can be multiplied together to generate a calculated concentration for each of the isotopes that correspond to the two or more measured peaks from the isotopic distribution of the calibrator for the analyte. In some embodiments, the known concentration of the calibrator for the analyte and either the theoretical relative abundance of the isotopes or the empirically derived abundance of the isotopes that correspond to three, four, five, six, seven, eight, nine, ten, eleven, twelve, thirteen, fourteen, fifteen, twenty, twenty-five, or thirty or more measured peaks in the isotopic distribution of the calibrator for the analyte can be multiplied together to generate a calculated concentration for each of the isotopes that correspond to the three, four, five, six, seven, eight, nine, ten, eleven, twelve, thirteen, fourteen, fifteen, twenty, twenty-five, or thirty or more measured peaks from the isotopic distribution of the calibrator for the analyte.

In some embodiments, the product of the known concentration of the calibrator for the analyte and either the theoretical relative abundance of the isotopes or the empirically derived abundance of the isotopes that correspond to the two or more measured peaks in the isotopic distribution of the calibrator for the analyte are the x-coordinates in the calibration curve. In some embodiments, the y-coordinates of the calibration curve are the signal intensities of the corresponding isotope in the isotopic distribution of the calibrator for the analyte.

In some embodiments, the calibration curve comprises three, four, five, six, seven, eight, nine, ten, eleven, twelve, thirteen, fourteen, fifteen, twenty, twenty-five, or thirty or more xy coordinates as described above. In some embodiments, the calibration curve from the calibrator for the analyte is generated by performing a linear regression analysis on the three, four, five, six, seven, eight, nine, ten, eleven, twelve, thirteen, fourteen, fifteen, twenty, twenty-five, or thirty or more xy coordinates. Examples of linear regression analyses include, but are not limited to, an unweighted Deming, a weighted Deming, a Passing-Bablok, an unweighted linear, and a weighted linear regression. In some embodiments, the calibration curve from the calibrator for the analyte is generated by performing a polynominal curve fit on the three, four, five, six, seven, eight, nine, ten, eleven, twelve, thirteen, fourteen, fifteen, twenty, twenty-five, or thirty or more xy coordinates.

In some embodiments, the calibration curve from the calibrator for the analyte is applied to at least one measured peak in the isotopic distribution of the analyte by performing a linear regression analysis.

In some embodiments, the linear regression analysis comprises solving the formula:

$$\frac{( Int - b ) m}{Theorectical Rel . Abun . of Isotope}$$

wherein Int is the signal intensity from a measured peak in the isotopic distribution of the analyte;
m is the slope of the calibration curve from the calibrator for the analyte; and
b is the y-intercept of the calibration curve from the calibrator for the analyte,

for at least one measured peak in the isotopic distribution of the analyte to yield an isotope concentration in the analyte. In some embodiments, the linear regression analysis is used to correct for variability in isotope pattern intensities between runs.

In some embodiments, the linear regression analysis comprises solving the formula:

$$\frac{( Int - b ) m}{Exp . Rel . Abun . of Isotope}$$

wherein Int is the signal intensity from a measured peak in the isotopic distribution of the analyte;
m is the slope of the calibration curve from the calibrator for the analyte; and
b is the y-intercept of the calibration curve from the calibrator for the analyte, for at least one measured peak in the isotopic distribution of the analyte to yield an isotope concentration in the analyte. In some embodiments, the linear regression analysis is used to correct for variability in isotope pattern intensities between runs.

In some embodiments, the calibration curve from the calibrator for the analyte is applied to at least one measured peak in the isotopic distribution of the analyte using a polynominal curve fit. For example, a polynomial curve fit can be used when the instrument response is not linear over the entire measurement range.

In some embodiments, the calibration curve from the calibrator for the analyte is applied to two or more measured peaks in the isotopic distribution of the analyte to yield two or more isotope concentrations for the analyte. For example, the calibration curve from the calibrator for the analyte is applied to two, three, four, five, six, seven, eight, nine, ten, eleven, twelve, thirteen, fourteen, fifteen, twenty, twenty-five, or thirty or more measured peaks in the isotopic distribution of the analyte to yield two, three, four, five, six, seven, eight, nine, ten, eleven, twelve, thirteen, fourteen, fifteen, twenty, twenty-five, or thirty or more isotope concentrations for the analyte. In some embodiments, all the isotope concentrations for the analyte (e.g., the two, three, four, five, six, seven, eight, nine, ten, eleven, twelve, thirteen, fourteen, fifteen, twenty, twenty-five, or thirty or more isotope concentrations for the analyte) are averaged to yield a concentration of the analyte in the sample.

### Calibrators and Analytes

An analyte can be a polypeptide, a protein, a peptide, a small molecule, a polysaccharide, a lipid, a glycan, a dendrimer, or a nucleic acid. In some embodiments, the analyte is a steroid. In some embodiments, the analyte is a molecule that is used in the diagnosis of one or more diseases. For example, the concentration of an analyte in a sample as determined using the methods described herein can be compared to the quantity of the analyte known to be associated with one or more diseases. In some embodiments, the analyte is a drug.

In some embodiments, the calibrator for the analyte is a polypeptide, a protein, a peptide, a small molecule, polysaccharide, a lipid, a glycan, a dendrimer, or a nucleic acid. In some embodiments, the calibrator for the analyte is a steroid. In some embodiments, the calibrator for the analyte is a drug. In some embodiments, the calibrator for the analyte is the same type of molecule as the analyte. For example, if the analyte is a polypeptide, the calibrator for the analyte is a polypeptide, or if the analyte is a small molecule, the calibrator for the analyte is a small molecule. In some embodiments, the nucleic acid is DNA. In some embodiments, the nucleic acid is RNA.

In some embodiments, the calibrator for the analyte is a variant of the analyte. For example, the calibrator for the analyte can be the same type of molecule as the analyte but have one or more differences such as the addition of one of more chemical groups, the removal of one or more chemical groups, or a substitution of one or more chemical groups for another (e.g., an amino acid substitution, a nucleic acid substitution, an isotopic substitution, an isobaric substitution, or an atomic substitution) as compared to the analyte. In some embodiments, one or more molecules (e.g., a mono- or polysaccharide) can be added to the calibrator for the analyte compared to the analyte.

In some embodiments, when the calibrator for the analyte and the analyte are both polymers (e.g., a polypeptide, a nucleic acid, a polysaccharide, or a glycan), the calibrator for the polymer has the same number of monomers as the analyte polymer. In some embodiments, when the calibrator for the analyte and the analyte are both polymers (e.g., a polypeptide, a nucleic acid, a polysaccharide, or a glycan), the calibrator for the analyte polymer can be a single monomeric variant of the analyte polymer. For example, when the calibrator for the analyte and the analyte are both polypeptides, the calibrator for the analyte polypeptide can be a single amino acid variant of the analyte polypeptide. In some embodiments, when the calibrator for the analyte and the analyte are both nucleic acids, the calibrator for the analyte nucleic acid can be a single nucleotide variant of the analyte nucleic acid. In some embodiments, when the calibrator for the analyte and the analyte are both polysaccharides, the calibrator for the analyte nucleic acid can be a single monosaccharide variant of the analyte polysaccharide.

In some embodiments, when the calibrator for the analyte and the analyte are both polymers (e.g., a polypeptide, a nucleic acid, a polysaccharide, or a glycan), the calibrator for the analyte polymer can consist of the same monomers as the analyte polymer, but have a different order of the monomers. For example, when the calibrator for the analyte and the analyte are both polypeptides, the calibrator for the analyte polypeptide can consist of the same amino acids as the analyte polypeptide, but have a different order of the amino acids. In some embodiments, when the calibrator for the analyte and the analyte are both nucleic acids, the calibrator for the analyte nucleic acid can consist of the same nucleotides as the analyte nucleic acid, but have a different order of the nucleotides. In some embodiments, when the calibrator for the analyte and the analyte are both polysaccharides, the calibrator for the analyte polysaccharide can consist of the same monosaccharides as the analyte polysaccharide, but have a different order or branching structure of the monosaccharides.

In some embodiments, the calibrator for the analyte has similar physical and/or chemical properties as the analyte. Such physical and/or chemical properties can include, but are not limited to, one or more of molecular weight, hydrophobicity, hydrophilicity, isoelectric point, chemical structure, polarizability, and ionization. For example, the calibrator for the analyte can have a molecule weight that is +/− about 25% of the molecular weight of the analyte. In some embodiments, the calibrator for the analyte can have a molecular weight that is within +/− about 20%, about 80%, about 85%, about 90%, about 95%, about 98%, about 99%, or about 100% of the molecular weight of the analyte.

In some embodiments, the calibrator for the analyte is an isobaric compound of the analyte.

In some embodiments, the calibrator has a different mass to charge ratio than the analyte (e.g., the mass to charge ratio as measured by a mass spectrometer).

In some embodiments, the calibrator is not isotopically labeled. For example, the calibrator does not contain an enriched isotope.

In some embodiments, the calibrator for the analyte is isotopically labeled. For example, the calibrator for the analyte can be labeled with ²H, ³H, ¹⁵N, ¹³C, ¹⁴C, ¹⁷O, ¹⁸O or a combination thereof, i.e., the concentration of ²H, ³H, ¹⁵N, ¹³C, ¹⁴C, ¹⁷O, ¹⁸O or the combination thereof can be enriched in comparison to the theoretical relative abundance of the isotope. In some embodiments, one, two, three, four, five, six, seven, eight, nine, ten, fifteen, twenty, thirty, forty, fifty or more of the H, N, C, or O atoms in the calibrator are isotopically labeled. In some embodiments, about 90% to about 100% of the H, N, C, or O atoms in the calibrator are isotopically labeled.

In some embodiments, the calibrator for the analyte is labeled with ²H, ¹⁵N, ¹³C, or a combination thereof via synthesis of the calibrator for the analyte using one or more isotopically labeled precursors. Other methods for isotopically labeling a calibrator for the analyte are described elsewhere, see, e.g., Ong et al. Mol. Cell Proteomics. 2002; 1(5):376-86; and Oda et al. Proc. Natl. Acad. Sci. U.S.A. 1999; 96(12): 6591-6596.

### Mass Spectrometry

Any mass spectrometry technique suitable for analyzing the type of analyte and/or calibrator for the analyte can be used in the methods described herein. For example, a mass spectrometer that can resolve at least two isotopic peaks of the analyte and at least two isotopic peaks of the calibrator for the analyte can be used. Non-limiting examples of mass spectrometry techniques suitable for use in the methods described herein include mass-spectrometry (e.g., single-stage mass spectrometry), tandem mass spectrometry (MS/MS), and sequential mass spectrometry (MSⁿ).

In some embodiments, the MS technique comprises the use of liquid chromatography (LC) or gas chromatography (GC). In some embodiments, the mass spectrometry technique comprises an LC-MS (liquid chromatography-MS) technique, a GC-MS (gas chromatography-MS) technique, or a MALDI-MS (matrix assisted laser desorption ionization-MS) technique. For example, in some embodiments, a sample as described herein can be extracted using an extraction column. In some embodiments, extraction can be followed by elution onto an analytical chromatography column. The columns can be useful, for example, to remove interfering components as well as reagents used in earlier sample preparation steps. Systems can be coordinated to allow the extraction column to be running while an analytical column is being flushed and/or equilibrated with solvent mobile phase, and vice-versa, thus improving efficiency and run-time. A variety of extraction and analytical columns with appropriate solvent mobile phases and gradients can be chosen by those having ordinary skill in the art. Analytes that elute from an analytical chromatography column can be then measured by mass spectrometry techniques such as those described herein or those described elsewhere. See, e.g., U.S. Pat. No. 10,712,336, which is herein incorporated by reference in its entirety.

In some embodiments, the mass spectrometry technique comprises the use of a single quadrupole mass spectrometer (Q), a triple quadrupole (QQQ) mass spectrometer, a linear ion trap mass spectrometer, a Q-linear ion trap mass spectrometer, a time of flight (TOF) mass spectrometer, a quadrupole-time of flight (Q-TOF) mass spectrometer, or a trapping mass spectrometer (Orbitrap or Fourier-transform ion cyclotron resonance (FTICR)). For example, a mass spectrometer with a resolution of 1:1000 can be used for a molecule (e.g., an analyte or the calibrator for the analyte) with a mass to charge ratio (m/z) of <1000. In some embodiments, a mass spectrometer with a resolution of 1:1000 can be used for a molecule (e.g., an analyte or the calibrator for the analyte) with a mass to charge ratio (m/z) of <500. In some embodiments, the MS technique comprises the use of high-resolution accurate mass-mass spectrometry (HRAM-MS).

In some embodiments, the MS technique is performed on lower resolution instruments for low molecular weight compounds.

### Samples and Sample Preparation

A sample for analysis can be any sample, including biological and non-biological samples. For example, a sample can be a biological sample, such as a tissue (e.g., adipose, liver, kidney, heart, muscle, bone, or skin tissue) or biological fluid (e.g., blood, serum, plasma, urine, lachrymal fluid, or saliva) sample. In some embodiments, the sample is from a tumor. The biological sample can be from a mammal. A mammal can be a human, dog, cat, primate, rodent, pig, sheep, cow, or horse. In some embodiments, a sample can be a food (e.g., a meat, dairy, or vegetative sample) or beverage sample (e.g., an orange juice or milk sample). In some embodiments, a sample can be a nutritional or dietary supplement sample. In some embodiments, the sample can be an environmental sample (e.g., a soil sample, water sample, or waste sample).

In some embodiments, the sample can be treated to remove components that could interfere with the mass spectrometry technique. A variety of techniques known to those having skill in the art can be used based on the sample type. Solid and/or tissue samples can be ground and extracted to free the analytes of interest from interfering components. In such cases, the sample can be centrifuged, filtered, and/or subjected to chromatographic techniques to remove interfering components (e.g., cells or tissue fragments). In some embodiments, reagents known to precipitate or bind the interfering components can be added. For example, whole blood samples can be treated using conventional clotting techniques to remove red and white blood cells and platelets. See also, e.g., Thomas et al. J Sep Sci. 2021 January; 44(1):211-246. In some embodiments, the sample can be treated as described herein before the calibrator is added to the sample. In some embodiments, the sample can be treated as described herein after the calibrator is added to the sample.

In some embodiments, the sample can be de-proteinized. For example, a plasma sample can have serum proteins precipitated using conventional reagents such as acetonitrile, KOH, NaOH, or others known to those having ordinary skill in the art, optionally followed by centrifugation of the sample. In some embodiments, de-proteinization comprises exposing the sample to acidified ethanol. Any appropriate method of polypeptide extraction or precipitation can be performed to decrease high abundance and high molecular weight polypeptides from a biological sample (e.g., a plasma or urine sample) prior to mass spectrometric analysis. In some embodiments, polypeptide purification can be performed to decrease high abundance and high molecular weight polypeptides from a biological sample (e.g., a plasma or urine sample) prior to mass spectrometric analysis. In some embodiments, acetonitrile polypeptide extraction/precipitation can be performed. In some embodiments, immunochemistry-based protein-depletion techniques can be performed to remove high abundance proteins from a biological sample. For example, commercially available kits such as the ProteoPrep® 20 (Sigma-Aldrich) plasma immunodepletion kit can be used to deplete high abundance proteins from plasma. In some embodiments, the sample can be de-proteinized before the calibrator is added to the sample. In some embodiments, the sample can be de-proteinized after the calibrator is added to the sample.

The invention will be further described in the following examples, which do not limit the scope of the invention described in the claims.

### EXAMPLES

### Example 1. Single-Substance Multi-Point Calibration

A multi-point calibration was developed that can use the relationship between the known concentration of a calibrator for an analyte, the theoretical relative abundance of isotopes of the calibrator for the analyte, and the signal intensity of the peaks corresponding to the isotopes in the mass spectrum of the calibrator for the analyte. The results of this method using insulin-like growth factor 1 (IGF-1) were found to be comparable to the reference IGF-1 test results.

The results of this method for cortisol measurements, as an example of a small molecule assay, were also found to be comparable to the cortisol reference test results.

### Materials and Methods

### Chemicals

Water was purified using a Barnstead Nanopure™ system (ThermoFisher Scientific). Hydrochloric acid and acetone were purchased from Fisher Scientific. Trizma base, LC-MS grade acetonitrile (ACN), formic acid, and isopropanol (IPA) were purchased from Millipore Sigma. Reagent grade ethanol was purchased from Brenntag Great Lakes.

### Serum Samples and Sample Preparation

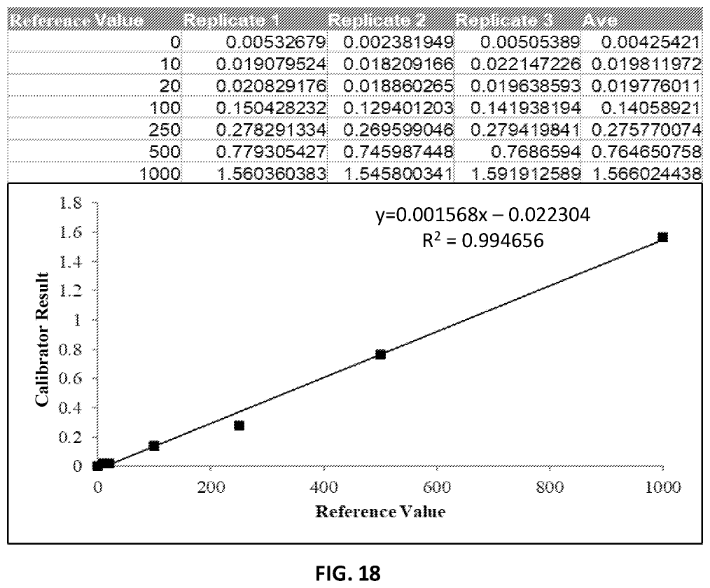

Recombinant wild-type IGF-1 was obtained from Cerilliant. Calibration accuracy was checked using IGF-1 World Health Organization International Standard material (National Institute for Biological Standards and Control). For the reference IGF-1 test, six calibration levels are used (10 ng/mL, 20 ng/mL, 100 ng/mL, 200 ng/mL, 500 ng/mL, and 1,000 ng/mL). Quality control samples were obtained by pooling excess serum samples. The internal standard (IS) for the reference IGF-1 method (IS; recombinant IGF-1 grown on ¹⁵N containing media) was sourced from Prospec Protein Specialists. The calibrator for the analyte for the single-substance multi-point calibration method was the recombinant IGF-1 variant A70T, which was also obtained from Prospec Protein Specialists. The calibrator value was determined based on the reference 6-point calibration curve. This process is shown in FIG. 18 for the ¹⁵N labeled data set. First, the calibrator linear regression correction was applied to the reference calibrators to yield a concentration relative to the calibrator. This was replicated 3 times and the average relative concentration was plotted versus the established reference calibrator concentrations. The resulting linear regression equation was then applied to each control and patient sample to yield a final result. Once this linear regression equation has been generated, there is no need for external calibration curves.

For all samples and quality control (QC) tests, 250 μL of internal standard or 100 μL of the calibrator for the analyte (depending on calibration technique) was added to 1000 μL of human serum. Next, 4000 μL of acidified ethanol, e.g., seven parts ethanol to one part 1N hydrochloric acid, was added to precipitate excess proteins and dissociate IGF-1 from binding proteins. The solutions were incubated for 30 min at room temperature. Subsequently, 90 μL of 1.5 M Tris was added to neutralize the solution, which was then centrifuged at 3,000 rpm for 10 min. This was followed by a 30 min incubation at −20° C. and centrifugation for 10 min at 3,000 rpm. The resulting supernatant then underwent LC-MS analysis.

### Liquid Chromatography-Mass Spectrometry Analysis

Liquid chromatography separation was performed using an Ultimate 3000 HPLC System (ThermoFisher Scientific) with a 2.0×4.0 mm C12 extraction cartridge (Phenomenex) and a 3.0×50 mm SB-C18 (Agilent Technologies) analytical column with 2.7 μm particles.

Mobile phase A was water with 0.1% formic acid and mobile phase B was 90% ACN, 10% water, and 1% formic acid. 40 μL of sample was loaded onto the extraction cartridge with a mobile phase of water and 0.1% formic acid. The loading pump was used to deliver 17% mobile phase B to the cartridge at 750 μL/min for 1 min. Next, the sample was eluted off of the cartridge at 150 μL/min for 2 min using 40% mobile phase B. This eluate was mixed via a tee with the eluting pump flowing 2% mobile phase B at 350 μL/min prior to loading onto the analytical column. After loading onto the analytical column, the percentage of mobile phase mobile phase B is increased to 23.5% over 15 sec and the sample is then separated with a linear gradient from 23.5% mobile phase B to 35.5% mobile phase B over 4 min at a flow rate of 500 μL/min. Mobile phase B is then ramped to 95% over 15 sec and is held there for 30 sec to wash the analytical column. The analytical column is re-equilibrated at 2% mobile phase B for 1.5 min. While performing separation and washing of the analytical column, the loading pump washes the trap cartridge for 1 min using a solution of 45% ACN, 45% IPA, and 10% acetone. This was followed by a 1 min wash at 100% mobile phase B and an additional wash at 40% mobile phase B for 1 min. The trap cartridge is then equilibrated at 17% mobile phase B for 2.25 min.

Mass spectrometry analysis was performed using a Q Exactive Plus mass spectrometer (ThermoFisher Scientific). The heated electrospray ionization (HESI) source utilized a spray voltage of 4 kV, a sheath gas flow rate of 60 psi, an auxiliary gas flow of 4 au, a sweep gas flow rate of 1 au, and a gas heater temperature of 360° C. All samples were analyzed by performing an MS scan from m/z 925-1145. The resolution for this analysis was set to 70,000 @ m/z 200, the maximum injection time (IT) into the Orbitrap was 200 ms, and the automatic gain control (AGC) target was 1×10⁶.

### Data Processing and Isotopic Calibration Calculations

Data processing for the reference IGF-1 test was performed using Tracefinder v.4.1. The quantitative results are based on summation of the five largest theoretical isotopes of the intact IGF-1 polypeptide to generate chromatograms for integration. Linear calibration curves are generated from calibrators daily and 1/× weighting is used. Single-substance multi-point calibration results were compared to the reference test via regression models using Analyse-It™ (Analyse-It Software). Calibration results were also compared to traditional methodologies via regression models using MS Excel 365.

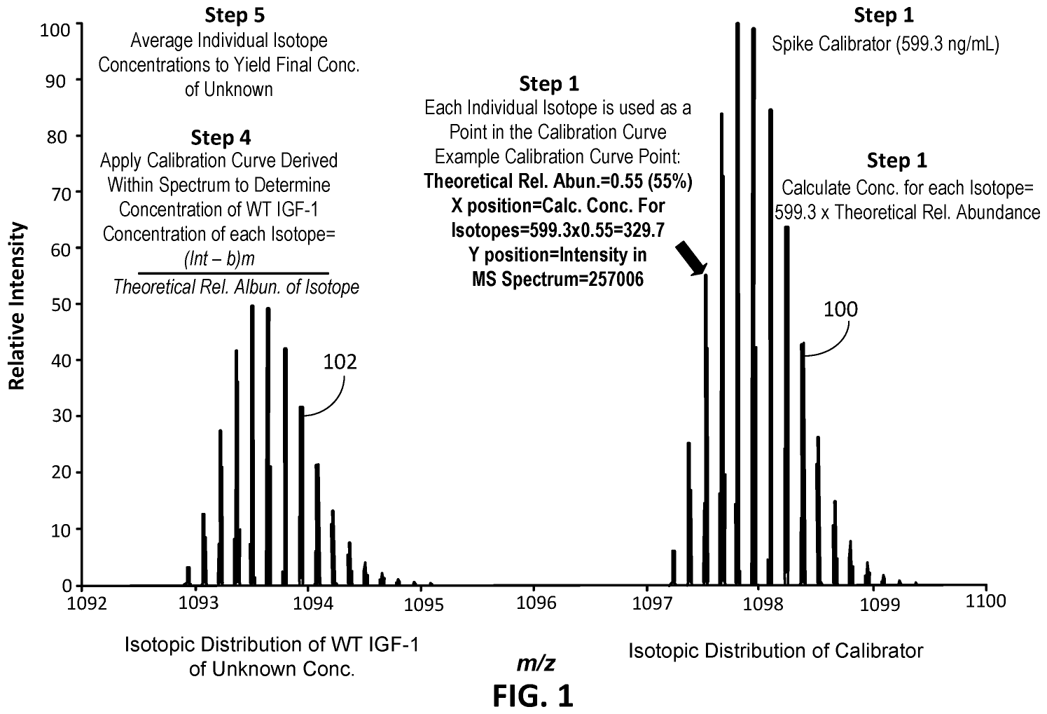

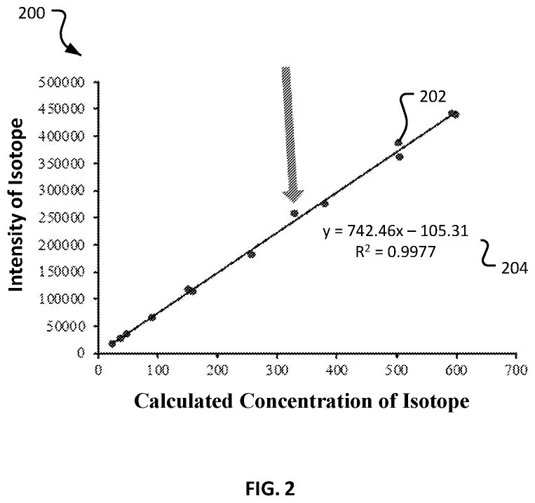

The general process for isotopic calibration is shown in FIG. 1. The calibrator for the analyte is spiked into each individual sample, and thereafter the sample is prepared and analyzed as in the reference IGF-1 test. Following LC-MS analysis, the spectra were opened in Xcalibur™ (ThermoFisher Scientific) and the spectra across the chromatographic peak were averaged. The spectral peak list from the averaged mass spectrum was exported into Microsoft Excel™ for mathematical manipulation. Theoretical relative isotopic abundances were generated with the Pacific Northwest National Laboratory Molecular Weight Calculator. A theoretical concentration for each isotope signal was then produced by multiplying the calibrator concentration (599.3 ng/mL) and the theoretical isotopic abundance. The results of this process are shown in FIG. 2. The x-coordinate in the isotopic calibration curve is the product of the concentration of the calibrator for the analyte and the theoretical relative abundance as shown. The y-coordinate was the detected signal intensity of the respective isotope. As shown in FIG. 2, this process resulted in thirteen calibration points 202 spanning from a theoretical concentration of 23.1-599.3 ng/mL. The calibration points 202 can then be fit with a line to generate the calibration curve. Referring again to FIG. 1, the calibration curve can then be applied to the wild-type (WT) IGF-1 signal 102 to determine the concentration of each signal intensity peak. This can be done by solving for x in the linear regression equation 204 and dividing that result by the theoretical relative abundance of the isotope. This can produce a concentration for each isotopic signal intensity peak, and the resulting 11 isotope concentrations for each signal intensity peak were averaged to produce a final concentration result. When the ¹⁵N labeled internal standard was used, the relative abundance of the calibrator for the analyte (equivalent to using the theoretical distribution) was established using 100 replicate measurements over the course of three days. Average relative isotope abundances from the 100 measurements were then used for the corrections thereafter.

### Samples/Sample Preparation-Cortisol and Cortisone

The external calibrators for cortisol and cortisone from the reference method were made from material purchased from Cerilliant. The reference method utilizes deuterated cortisol (Cat #-S7193-0.1; 4 deuteriums) and cortisone (Cat #-S8056-0.1; 8 deuteriums) obtained from IsoSciences (Ambler, Pa., USA). Quality control samples were prepared by spiking calibration material into stripped urine. Both the deuterated compounds from IsoSciences and ¹³C labeled cortisol (Cat #-CLM-10371-C, Cambridge Isotope Labs, Tewksbury, Mass., USA) and cortisone (CLM-10536-C, Cambridge Isotope Labs) were tested as isotopic distribution calibrators. Both labeled internal standard materials were inadequately pure to be used gravimetrically as calibration material. Therefore, the calibrator values were determined based on the established 6-point calibration curve. Urine samples were prepared by placing 100 μL of sample and 100 μL of IsoC into a 96 well plate, followed by mixing for 10 mins.

### Liquid Chromatography-Mass Spectrometry Analysis-Cortisol and Cortisone

The instrument hardware for these analyses was as described above in the IGF-1 methods section. Sample (50 μL) was injected onto a Cohesive Technologies TurboFlow™ HTLC C18 XL (ThermoFisher Scientific, Cat #CH-953280). Mobile Phase A was water with 0.1% formic acid and mobile phase B was methanol with 0.1% formic acid. The sample was loaded and washed at 0% B at 1.5 mL/min and then washed off the TurboFlow column at 50% B and 0.25 mL/min. The sample was diluted with 10% B at 0.75 mL/min and focused on an Agilent Technologies Zorbax™ XDB-C18 (Cat #-935967-902) analytical column. Chromatographic separation was then done via an isocratic method at 55% B. Columns were then washed and equilibrated over the course of 2.5 min. These MS analyses were also conducted with a Q Exactive Plus mass spectrometer operated in full scan MS mode scanning from m/z 355 to 372.5. The resolution was 140,000 the AGC target was 1×10⁶, and the maximum injection time was 200 ms. The HESI source utilized a spray voltage of 4 kV, a sheath gas flow rate of 35 psi, an auxiliary gas flow of 10 au, a sweep gas flow rate of 5 au, and a gas heater temperature of 350° C.

### Data Processing and Isotopic Calibration Calculations—Cortisol and Cortisone

Results were compared to clinical reports generated using a Sciex (Framingham, Mass., USA) 5000 triple quad mass spectrometer operated in multiple reaction monitoring (MRM) mode. As above, the isotopic distribution calibrator was spiked into each individual sample in place of the traditional IS and no modifications were made to the sample preparation procedure. The spectral peak list was generated as above. The relative abundances for the detectable isotopes were established using 100 replicate measurements. The calculated concentration results were generated using the mathematical process outlined above.

### Results and Discussion

### Calibration for IGF-I Quantification

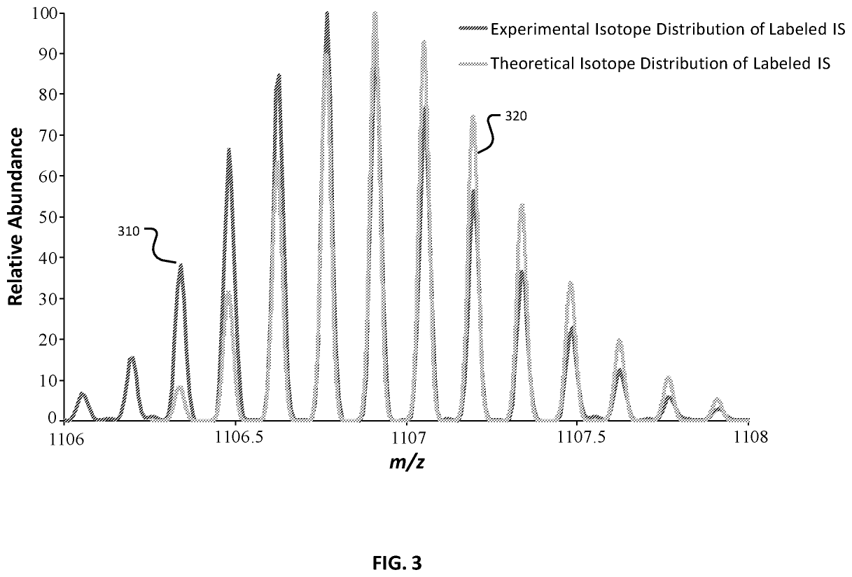

The reference IGF-1 test uses ¹⁵N labeling, which was initially tried as the calibrator for the analyte in the single-substance multi-point calibration method. As shown in FIG. 3, the detected isotopic distribution of ¹⁵N-labeled IFG-1 (310, shown in dark gray) has a lower m/z than the predicted theoretical isotopic distribution (320, shown in light gray). This can be indicative of incomplete incorporation of ¹⁵N into the IGF-1.

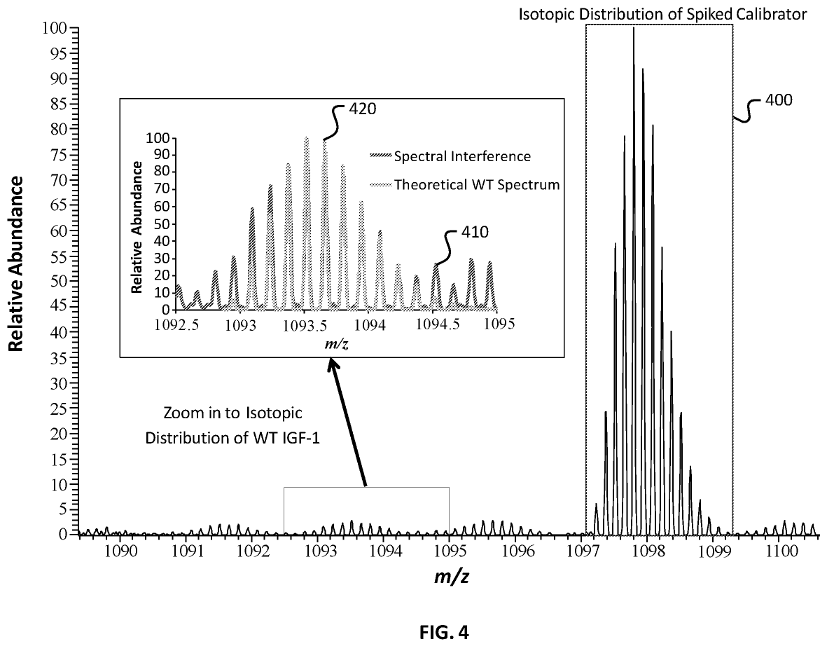

The viability of a single amino acid variant of IGF-1 (e.g., A70T) as a calibrator for the analyte was tested. Single amino acid variants can produce consistent isotopic distributions and can have similar chemical and physical properties to the wild-type polypeptide. One issue encountered when using A70T as the calibrator for the analyte is shown in FIG. 4. FIG. 4 depicts a mass spectrum showing the relative abundance in the isotopic distribution of A70T with respect to the m/z ratio. The isotopic relative abundance signal peaks of A70T can be seen in box 400 with an average m/z of about 1097.8. Inset to FIG. 4 shows the theoretical relative abundance signal isotopic peaks of WT IGF-1 with an average m/z of about 1093.5. The difference in average m/z between A70T and WT IGF-1 was relatively small with an average m/z difference of about 4. At this difference, artifacts of the A70T signal peaks 410 can interfere with the WT IGF-1 signal peaks 420. One method to overcome this interference between the pictured m/z ranges of 1092 through 1095 (420) can include subtracting out the calculated value, mitigating the impact of this interference on the calculated values. Although an isotopically labeled IGF-1 polypeptide with a larger m/z shift can reduce interference further, it was determined that single amino acid variant A70T was suitable for these experiments.

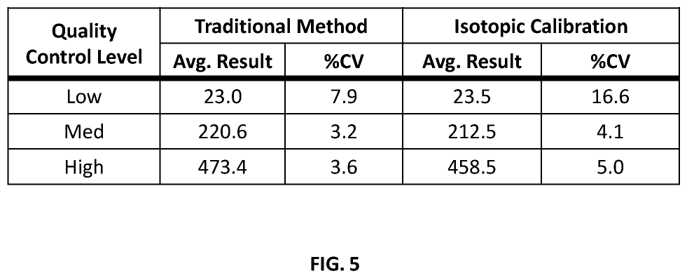

Following selection of the calibrator for the analyte, ten experiments were performed over the course of 10 days. Precision results from twenty QCs of each level (two per experiment) are shown in FIG. 5. When using isotopic calibration, the percent coefficient of variation (% CV) was considered acceptable (<20%) for all levels; the % CV was approximately 5% for the medium and high level, but was higher near the lower limit of quantitation (LLOQ).

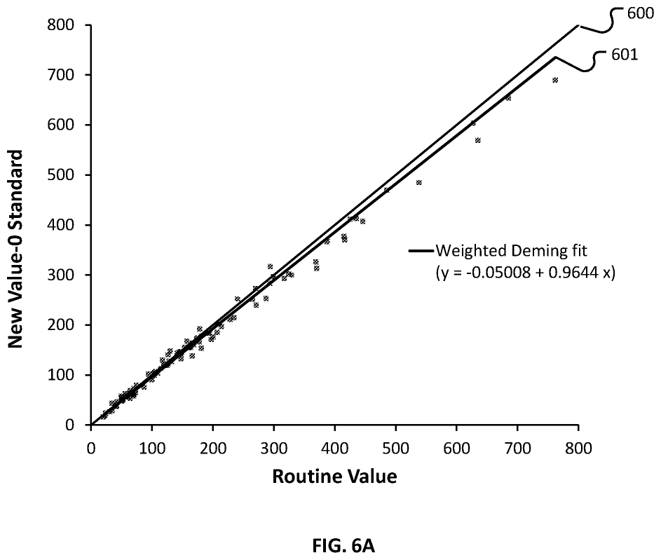

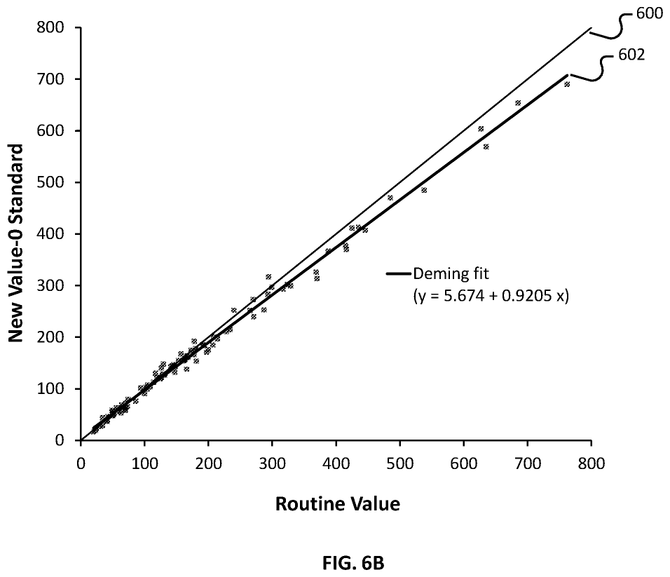

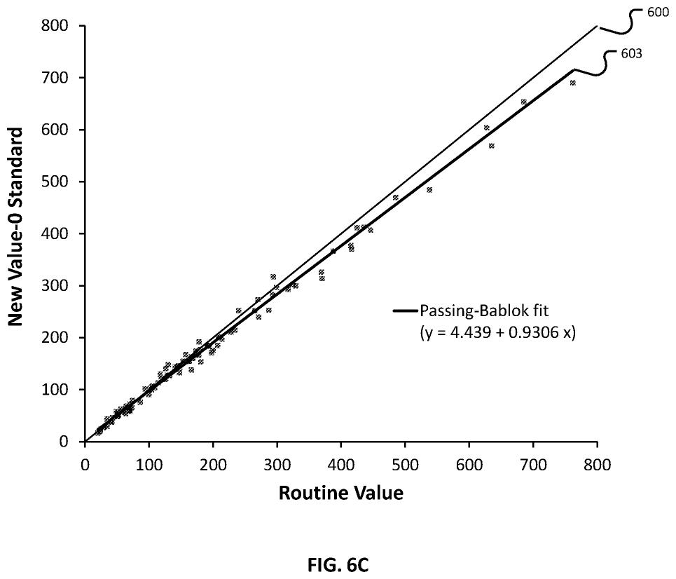

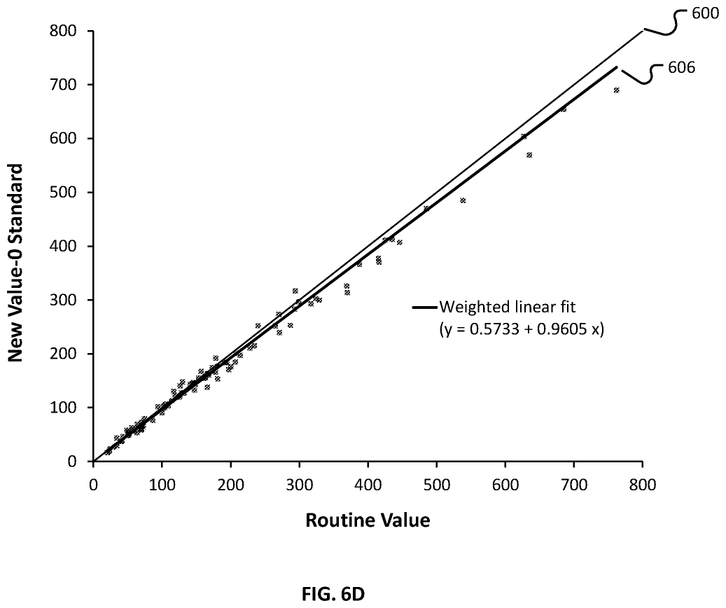

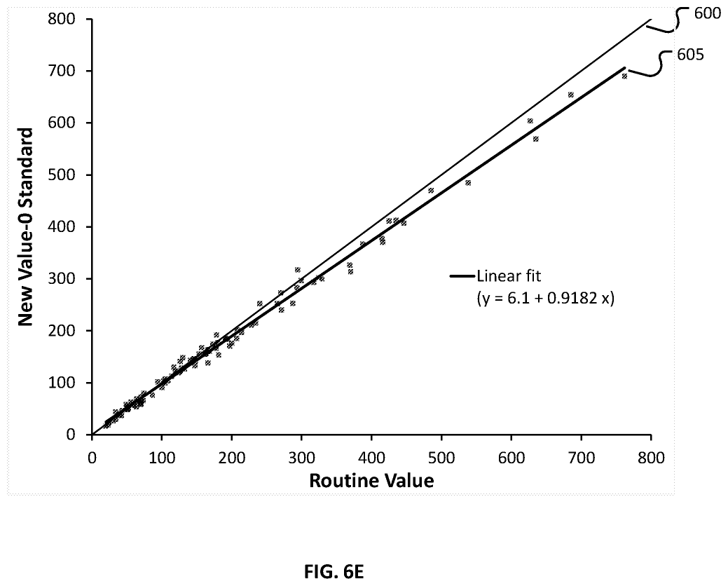

In addition to precision, an accuracy comparison was also conducted (10 samples per day, 100 total). These results were fitted to several variations of linear regression analyses including an unweighted Deming, a weighted Deming, a Passing-Bablok, an unweighted linear, and a weighted linear regression. As can be seen in FIG. 6A, the patient results from the single-substance multi-point calibration fitted using the Weighted Deming regression 601 were comparable to the reference IGF-1 test 600. Alternative linear regression analysis models are shown in FIGS. 6B-6E including an unweighted Deming linear regression analysis model (FIG. 6B, 602), a Passing-Bablok linear regression analysis model (FIG. 6C, 603), a weighted linear regression analysis model (FIG. 6D, 604), and an unweighted linear regression analysis model (FIG. 6E, 605), respectively.

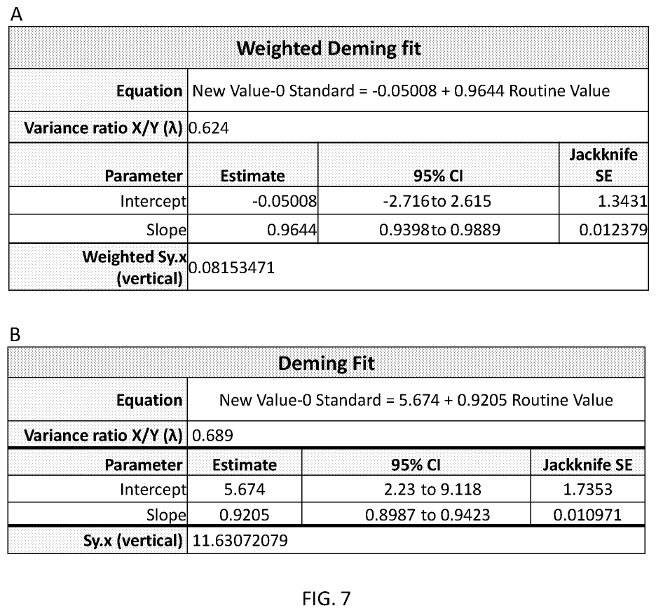

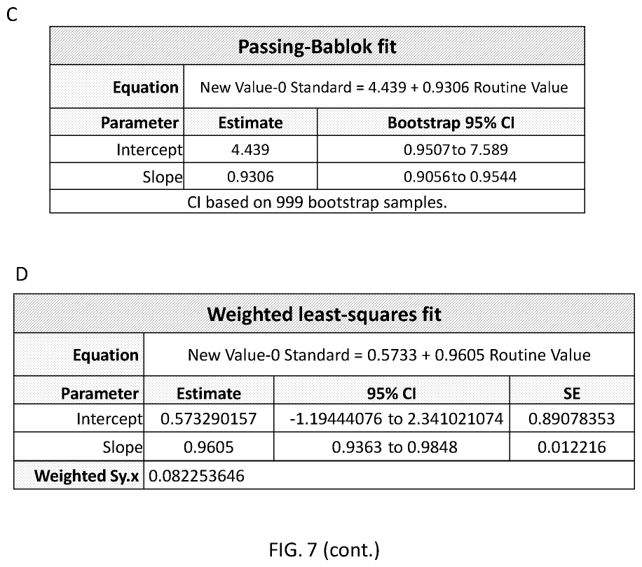

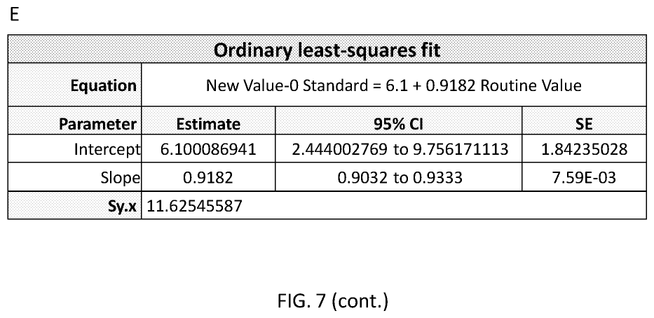

The goodness-of-fit statistics from the linear regression analysis models were considered acceptable and are shown in FIGS. 7A-7E. Generally, each linear regression analysis model outputs an estimated intercept and slope values based upon weighting factors associated with each linear regression analysis model. Along with the estimated values, estimates of the goodness of fit are also output and shown as 95% confidence levels (CL) for each linear regression analysis models. The 95% CLs are based upon 999 bootstrapped samples. The average percent difference between the reference method and the single-substance multi-point calibration results was 3.4% (not shown), which further indicated the near equivalence of these methods.

The statistics from all of the regression models were considered acceptable. Additionally, the average percent difference between the established method and the isotopic calibration results was 3.4%, which further indicated near equivalence of the techniques for producing quantitative results.

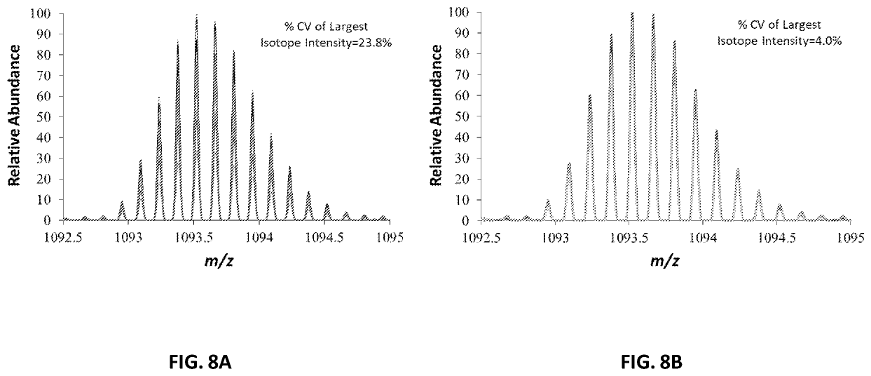

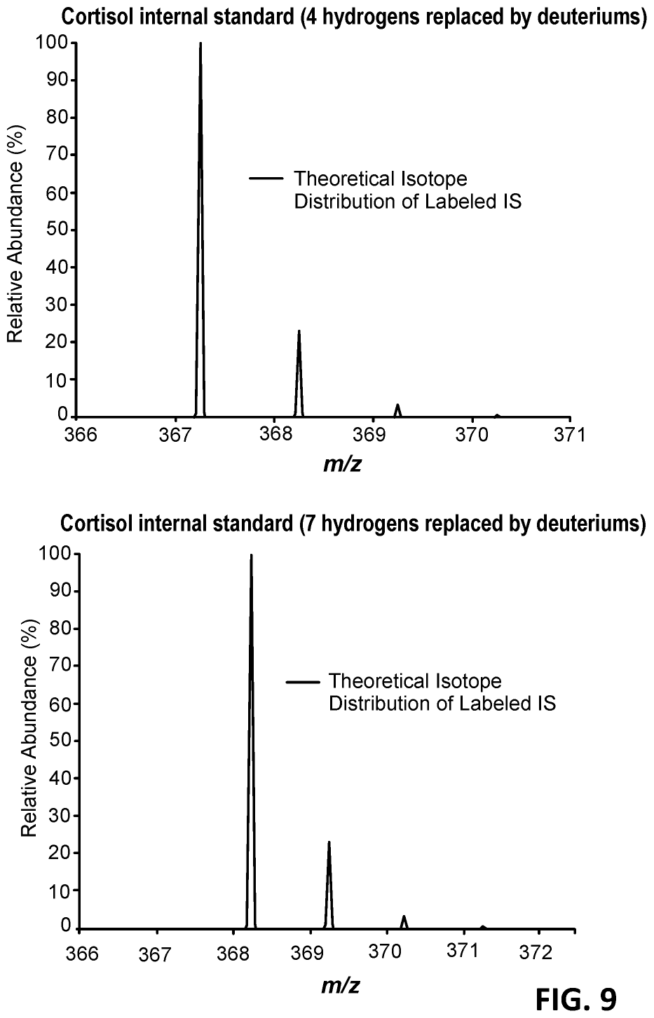

An impact of applying the isotopic calibration linear regression correction determined using the linear regression correction in FIG. 2 to experimentally derived spectra is shown in FIG. 8. FIG. 8A depicts the uncorrected isotopic distribution of the WT IGF-1 at the medium QC concentration level. FIG. 8B shows the overlaid isotopic distribution of the WT IGF-1 after correction applying the linear regression correction. Prior to applying the correction, the signals were highly variable (% CV=23.8% of largest isotope signal); however, once the correction was applied the signals were much more consistent (% CV=4.0% of largest isotope signal). All of the calibrator experiments used 599.3 ng/mL of IGF-1 A70T as the calibrator. Without linear regression correction (FIG. 8A), there was considerable variability in ion intensity between the different distributions, which was almost completely corrected after linear regression (FIG. 8B).

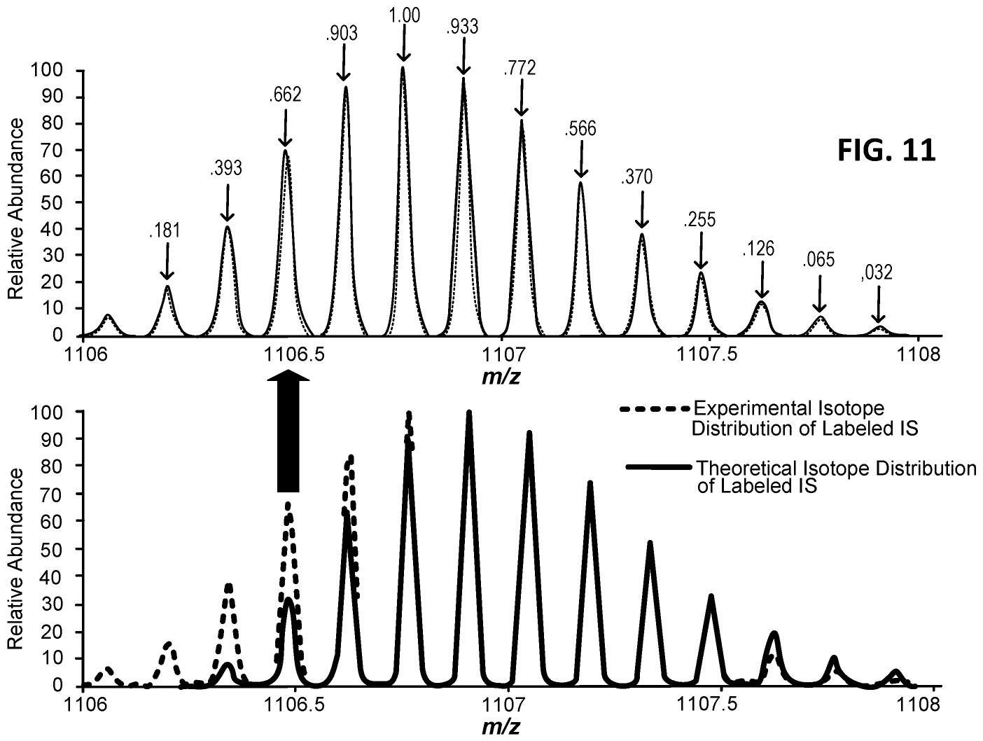

The IGF-I ¹⁵N-labeled internal standard was further tested as a calibrator. This involved some correction as the detected isotopic distribution showed a lower average mass than predicted, indicative of incomplete ¹⁵N incorporation, as noted above (see also FIG. 3). This was corrected by normalizing the relative abundance by averaging the relative abundances from 100 replicate sample preparations and LC-MS measurements (see FIG. 11). A relative abundance for each of the isotopes was produced from every replicate and the average relative abundance from the 100 replicate measurements was used for generating the calibrator linear regression curve as shown in FIG. 1.

This methodology can be used to make use of any consistent isotopic distribution as a calibrator. Closely related molecules are more likely to correct for variables such as ion suppression.

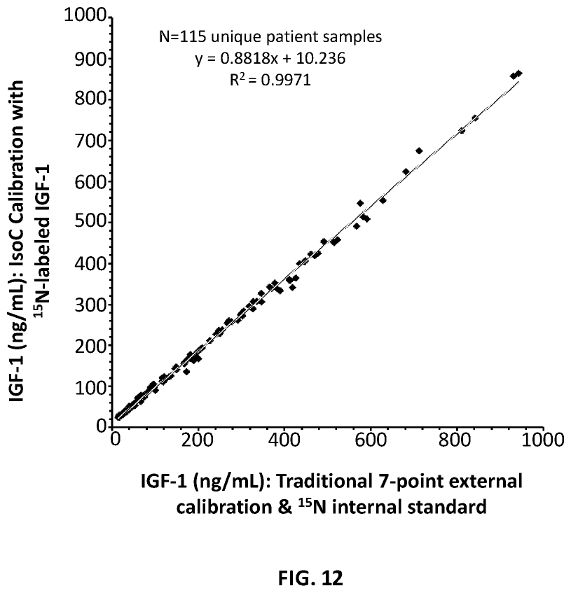

When the results of patient samples that had been run with the routine, IS-corrected seven-point external calibration curve were compared with the ¹⁵N calibrator calibration, a good agreement of the results was found (see FIG. 12).

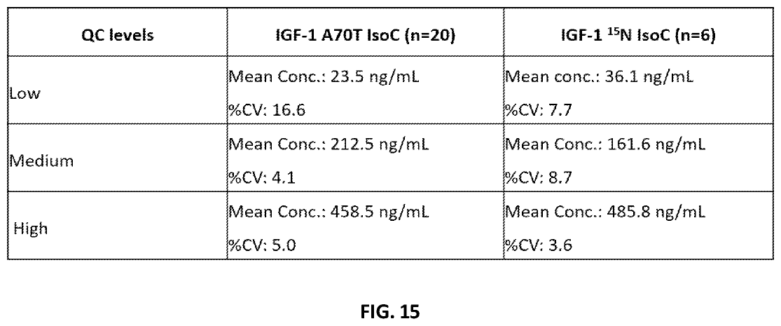

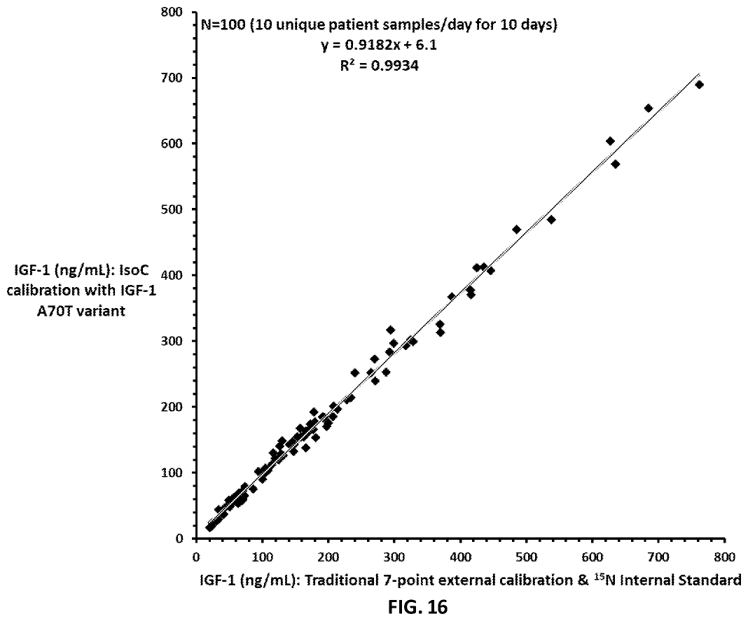

Linear regression analyses comparing the reference method to isotopic distribution calibration using the A67T and 15N labeled IGF-1 can be seen in FIG. 16 and FIG. 12 respectively. The statistics from these linear regression analyses was considered acceptable and demonstrated strong agreement between the two techniques. Day to day precision results are shown in FIG. 15.

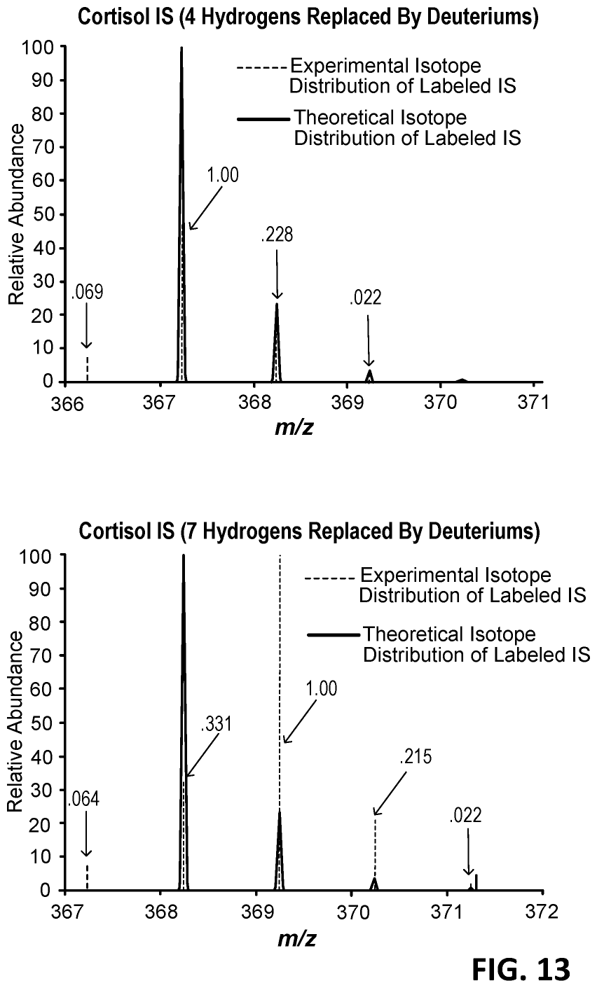

Calibrator calibration for simultaneous cortisol and cortisone quantification Both deuterated and ¹³C-labeled cortisol and cortisone were tested as calibrators. The observed isotope distribution for the deuterated cortisol and cortisone calibrators are shown in FIG. 13. The experimental isotope distributions did not align with expected theoretical distributions, but the distributions were consistent over replicate measurements, and relative abundances were assigned by averaging the intensities across the replicate measurements.

For example, the cortisone IS appeared to have 8 deuterium residues in contrast to the manufacturers' stated composition. However, by using the replicate measurement methodology described herein to assign relative abundance, these molecules were still able to be used as calibrators. In fact, it was possible to make use of the detected apparent exchange artifacts at lower m/z as the relative abundance was consistent.

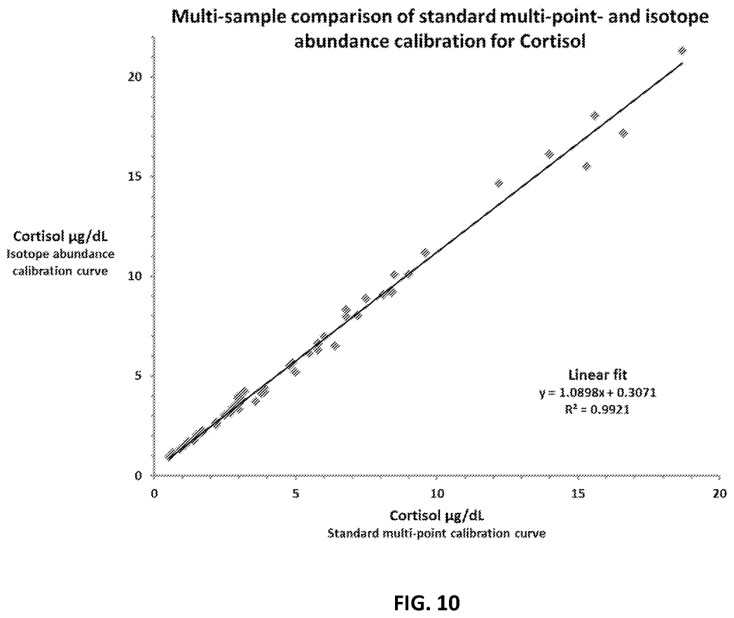

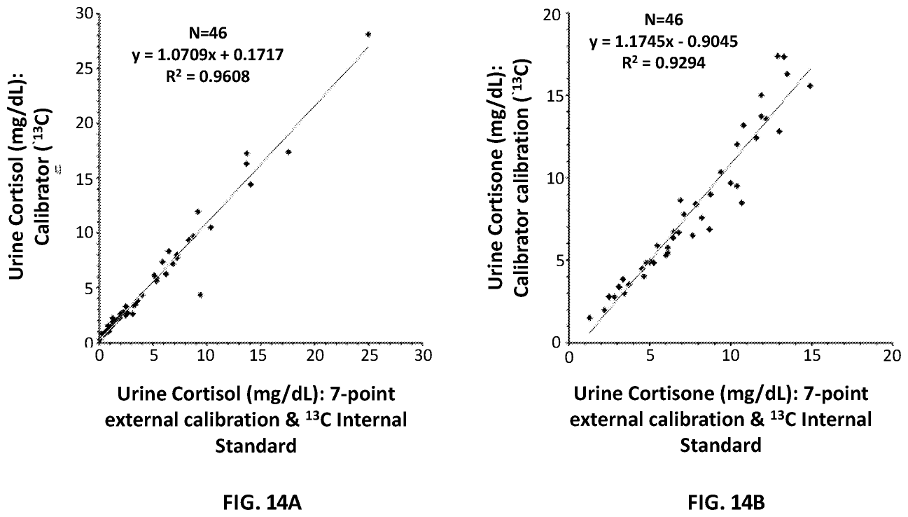

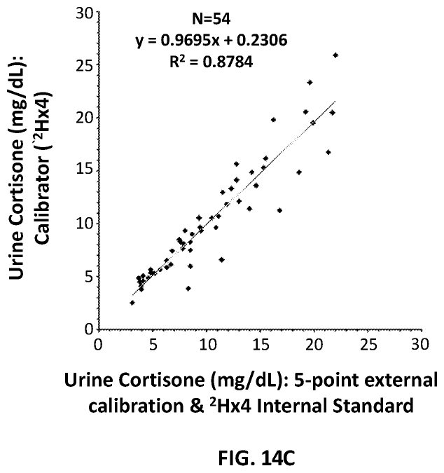

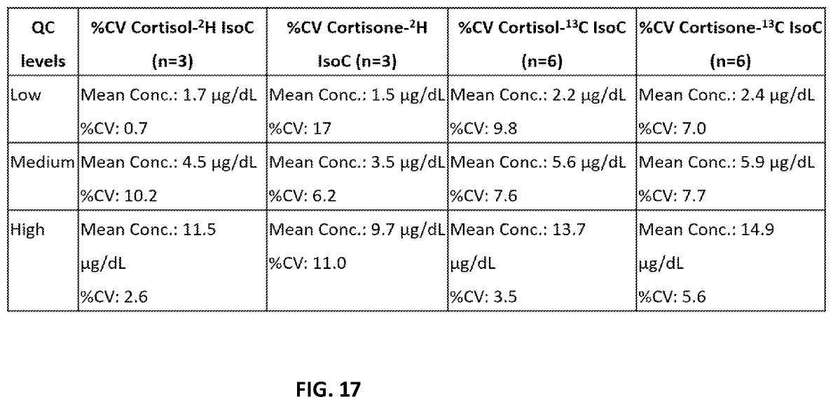

Comparison of patient results between calibrator calibration and standard external calibration after correction of the isotopic distribution yielded a reasonable linear fit for cortisol and cortisone (see FIGS. 14A-C and FIG. 10). Imprecision of the calibrator results for cortisone and cortisol are shown in FIG. 17.

### Other Embodiments

A number of embodiments of the invention have been described. Nevertheless, it will be understood that various modifications may be made without departing from the spirit and scope of the invention.

1. A method for determining the concentration of an analyte in a sample comprising the analyte and one calibrator for each analyte, wherein the concentration of each calibrator is known, the method comprising:
detecting the analyte and the calibrator for the analyte in the sample using a mass spectrometry (MS) technique; and
determining the concentration of the analyte in the sample based on two or more peaks in the isotopic distribution of the calibrator for the analyte.

2. The method of claim 1 wherein the calibrator for the analyte is isotopically labeled.

3. The method of claim 1, wherein the calibrator for the analyte is not isotopically labeled.

4. The method of claim 1, wherein determining the concentration of the analyte comprises generating a calibration curve based on:
a) the known concentration of the calibrator for the analyte;
b) the theoretical relative abundance of the isotopes that correspond to the two or more peaks from the isotopic distribution of the calibrator for the analyte; and
c) the signal intensity of the two or more peaks in the isotopic distribution of the calibrator,
wherein the known concentration of the calibrator for the analyte and the theoretical relative abundance of the isotopes that correspond to the two or more peaks in the isotopic distribution of the calibrator for the analyte are multiplied together to generate a calculated concentration for each of the isotopes that correspond to the two or more peaks from the isotopic distribution of the calibrator for the analyte.

5. The method of claim 4, wherein the calibration curve from the calibrator for the analyte is applied to at least one peak in the isotopic distribution of the analyte by performing a linear regression analysis.

6. The method of claim 5, wherein the linear regression analysis comprises solving the formula:
$$\frac{( Int - b ) m}{Theorectical Rel . Abun . of Isotope}$$
wherein Int is the signal intensity from a peak in the isotopic distribution of the analyte;
m is the slope of the calibration curve from the calibrator for the analyte; and
b is the y-intercept of the calibration curve from the calibrator for the analyte,
for at least one peak in the isotopic distribution of the analyte to yield an isotope concentration in the analyte.

7. The method of claim 5, wherein the calibration curve from the calibrator for the analyte is applied to two or more peaks in the isotopic distribution of the analyte to yield two or more isotope concentrations for the analyte.

8-11. (canceled)

12. The method of claim 1, wherein the analyte is a polypeptide, a protein, a peptide, a small molecule, polysaccharide, lipid, dendrimer, glycan, or a nucleic acid.

13. The method of claim 1, wherein the calibrator is a polypeptide, a protein, a peptide, a small molecule, polysaccharide, lipid, dendrimer, glycan, or a nucleic acid.

14. The method of claim 1, wherein the calibrator is a variant of the analyte.

15. The method of claim 1, wherein the calibrator has a different mass to charge ratio than the analyte.

16-35. (canceled)

36. A method for determining the concentration of an analyte in a sample comprising the analyte and one calibrator for each analyte, wherein the concentration of each calibrator is known, the method comprising:
detecting the analyte and the calibrator for the analyte in the sample using a mass spectrometry (MS) technique; and
determining the concentration of the analyte in the sample based on two or more peaks in the isotopic distribution of the calibrator for the analyte;
wherein determining the concentration of the analyte comprises generating a calibration curve based on:
a) the known concentration of the calibrator for the analyte;
b) the experimentally derived relative abundance of the isotopes that correspond to the two or more peaks from the isotopic distribution of the calibrator for the analyte; and
c) the signal intensity of the two or more peaks in the isotopic distribution of the calibrator,
wherein the known concentration of the calibrator for the analyte and the theoretical relative abundance of the isotopes that correspond to the two or more peaks in the isotopic distribution of the calibrator for the analyte are multiplied together to generate a calculated concentration for each of the isotopes that correspond to the two or more peaks from the isotopic distribution of the calibrator for the analyte.

37. The method of claim 36, wherein the calibrator for the analyte is isotopically labeled.

38. The method of claim 36, wherein the calibrator for the analyte is not isotopically labeled.

39. The method of claim 36, wherein said generating a calibration curve is further based on the theoretical relative abundance of the isotopes that correspond to the two or more peaks from the isotopic distribution of the calibrator for the analyte.

40. The method of claim 36, wherein the calibration curve from the calibrator for the analyte is applied to at least one peak in the isotopic distribution of the analyte by performing a linear regression analysis.

41. The method of claim 40, wherein the linear regression analysis comprises solving the formula:
$$\frac{( Int - b ) m}{Exp . Rel . Abun . of Isotope}$$
wherein Int is the signal intensity from a peak in the isotopic distribution of the analyte;
m is the slope of the calibration curve from the calibrator for the analyte; and
b is the y-intercept of the calibration curve from the calibrator for the analyte,
for at least one peak in the isotopic distribution of the analyte to yield an isotope concentration in the analyte.

42. The method of claim 40, wherein the calibration curve from the calibrator for the analyte is applied to two or more peaks in the isotopic distribution of the analyte to yield two or more isotope concentrations for the analyte.

43. The method of claim 36, wherein the calibration curve from the calibrator for the analyte is applied to three or more peaks in the isotopic distribution of the analyte to yield two or more isotope concentrations for the analyte.

44-70. (canceled)
